(19) United States
(12) Patent Application Publication
(10) Pub. No.: US 2022/0370853 A1
(43) Pub. Date: Nov. 24, 2022

(54) J-SLEEVE SYSTEM

(71) Applicant: Jeremy Henderson, Richmond, TX (US)

(72) Inventor: Jeremy Henderson, Richmond, TX (US)

(21) Appl. No.: 17/452,685

(22) Filed: Oct. 28, 2021

Related U.S. Application Data
(60) Provisional application No. 63/106,447, filed on Oct. 28, 2020.

(51) Int. Cl. A63B 24/00; A41D 13/00; A63B 71/06

(52) U.S. Cl. CPC A63B 24/0075; A41D 13/0015; A63B 71/0622; A63B 2225/50; A63B 2225/02; A63B 2024/0068

(57) ABSTRACT

The J-Sleeve System provides a system for collecting and analyzing data from the athletic performance of an athlete to provide feedback and advice on preferred movements to produce increased accuracy and efficient in the performance of the athlete and to improve understanding of the competitive landscape. Users would wear the J-sleeve on their arm during workouts, games, back yard skill sessions for which data is desired for analysis with JSS to improve efficiency and performance of user. JSS uses data and artificial intelligence to improve the efficiency of the athletes and their athletic performance. JSS capabilities include athletic movement form replay and accuracy, movement location tracking, fatigue level tracking, social sharing and competition, and a virtual clipboard for developing plays and positioning, and video game integration. The virtual clipboard allows users to draft optimal plays using data to determine the user with the best chance of success.

### FIELD OF INVENTION

The present disclosure relates generally to a system for preserving the mechanical aspects of human movement for analysis particularly as it relates to skill specific activities in sports, physical rehabilitation, and general athletic actions.

### BACKGROUND

Athletes consistently and continuously practice their sport in an effort to achieve perfection and maximum efficiency and consistency during sports competitions. They also put their bodies through strenuous physical rehabilitation and recovery regimes to achieve and maintain peak performance. Similarly, non-sports athletes pursue peak physical health by engaging in general exercise techniques. Coaches may assist the sports athletes in achieving their goals by providing analysis and critique of an athlete's performance and skill set. Feedback or comments from a coach or staff person are typically based on the coach's visual observations and notes from the athlete's practice or game session. Coaches may also rely on video recordings or the visual observations of other coaching staff. Currently, when coaches and athletes want to determine what movements are required for perfect athletic performance, they may depend on the observation of the coach or trainer and the recommendations of the coach or trainer. The athlete may also attempt to self-diagnose any issues preventing perfect performance. Both of these options are limited by human error and the ability to receive and process those visual observations. These methods are not capable of acquiring and analyzing a volume of data comparable to the abilities of a computer or artificial intelligence system. In addition, compression sleeves are typically used for the treatment or prevention of injuries and do not collect data or provide feedback on activities pursued to achieve a desired result specific to the sport or activity. These products may not be customized to the end user and may not provide ongoing insights for improvement specific to the activity performed. These existing systems may not provide analysis of functionality of form for accelerated efficiency.

The present J-Sleeve System (JSS) leverages artificial intelligence to aid the athlete in achieving perfect performance by increasing efficiency and accuracy. JSS allows parents, coaches and athletes to track the skill development and physical activity of the athletes, provides a training and analytical tool to enhance skill development for youth athletes, and showcases the athlete's skill profile that is accessible to recruiters and coaches. JSS capabilities include shot form replay and accuracy, shot location tracking, fatigue level tracking, social sharing and competition, and a virtual clipboard for developing plays, positioning and optimizing game plan strategy, and video game integration.

JSS is customized to the end user, who may be the athlete, parent, coach, staff, or other recipient user of JSS, and provides ongoing insights for improvement, which accelerates the time for the athlete to increase efficiency and achieve perfection or near-perfection. JSS offers continued learning opportunities with varying levels of competency and may be tailored to users in different sports fields such as basketball, golf, baseball, football, soccer, hockey or even physical therapy and general physical health. JSS provides a method of self-capturing a digital rendering of a replay image of the mechanical aspects of a jump shot or other human athletic movement for analytic consumption. The JSS helps close the gap in understanding how to improve the efficiency and accuracy of the athlete's jump shot.

### OBJECTIVES

An object of one embodiment of the present disclosure is to provide biometric data for diagnostic analysis of an athlete's movements.

Another object of the present disclosure is to provide a system for manual tracking of an athletes' movements during performance and positioning of the athlete on a field of court for the optimization of game planning and training strategies.

Another object of the present disclosure is to provide real-time and feedback and analysis of an athlete's movements to the athlete, coach and trainer.

Another object of the present disclosure is to provide post-process analysis by storing data and sending said data to a user after the completion of a performance or session.

Another object of the present disclosure is to provide a system for the comparison of performance data of multiple athlete users.

Another object of the present disclosure is to provide a durable, moisture resistant and wearable data collection garment or device.

Another object of the present disclosure is to provide a visual interface for the review, analysis, and comparison of data collected from an athlete.

Another object of the present disclosure is to provide a social platform for user-to-user competition from the data collected from an athlete.

Another object of the present disclosure is to provide a leader board that tracks and compares the data and analysis collected from an athlete against other athletes in the data set.

### SUMMARY

Embodiments of the present disclosure, the J-Sleeve System (JSS), generally provide a system for collecting and analyzing data from the athletic performance of an athlete to provide feedback and advice on preferred movements to produce increased accuracy and efficient in the performance of the athlete. JSS helps close the gap in understanding how to improve the efficiency of the athletes jump shot. The JSS also provides exposure to understand the competitive landscape. The invention claimed here solves this problem. JSS capabilities include shot form replay and accuracy, shot location tracking, foot positioning, leg movement, fatigue level tracking, social sharing and competition, and a virtual clipboard for developing plays and positioning, and video game integration. The virtual clipboard allows users and coaches to draft plays using player data. Users would wear the JSS on their arm during workouts, games, back yard skill sessions for which data is desired for analysis with JSS to improve efficiency and performance of user. JSS may also be used in other sports and physical activities where technique is critical to achieving desired outcomes, such as golf, baseball, physical therapy, football, soccer, hockey or general health and wellness.

JSS is a combination of hardware and software elements. JSS hardware may include: electronic compression sleeve, accelerometer, gyroscope, pressure sensor, heart rate monitor, short-range wireless communication transmitter (Bluetooth, for example), and a power source. The data captured with the JSS hardware is processed by JSS custom software code for further processing and analytics. JSS uses sensor technology with the compression sleeve to capture data, which is aggregated on a board with a battery pack and response mechanism. Sealing is poured over the board to case in sensors and protect them from water damage. Once activated, the sensors store data and synchronize with the mobile device via wireless communication transmitter. The mobile device post processes the data to provide custom analytics for end user consumption. JSS software processes collected data with if/then relationships to eliminate false positive readings, identify shot form, and store user metrics. JSS hardware captures data specifically related to the technique of which the user desires to analyze, such as shooting a basketball for the shooting arm only in this embodiment. The collected data is pushed to the cloud, stored, cut, drawn down and JSS analytics are calculated based on this data. JSS hardware is lightweight enough to be worn without hindering or impeding the wearer, durable enough to withstand the rigors of daily athletic use, moisture resistant with sufficient battery life to last throughout hours of use. JSS software filters the collected data needed to draw insights from a customized JSS library to give feedback on the athlete's technique. The customized data in the JSS library is collected from extensive research and collection of data points from many users to determine the ideal movements and speed of a particular athletic movement for a particular sport. In the case of the basketball jump shot, JSS would focus on the three main phases of shooting a jump shot, for example: the shooting pocket, stability and follow through. The JSS data analytics and digital rendering outline the specific capabilities that would allow the user to optimize their post workout reflection time on areas for improvement.

The present embodiment of JSS is focused on the mechanics of a basketball player but the system may be adapted to other athletes and sports. The present embodiment of JSS is a wearable electronic sleeve that a user would place on the user's arm. The JSS is designed to track and report the shot mechanics and biomarkers of the user in real time. In the instance of a jump shot, over 150 data points may be collected per shot and sent to a custom JSS mobile application that stores and reports the data. Among the data categories are shot performance, mechanics/form, and heart rate, however, data categories may be altered depending on the sport and movements being analyzed by JSS. From this data, the J-Sleeve determines the parameters of the ideal movement, “Golden Template”, comprised of the best shots according to the player and/or the coach, which is the three best shots in this example. JSS then compares every shot from the user thereafter to the data points from the Golden Template, and a performance percentage is assigned to the user for that movement. To capture and transmit these data points, the core JSS hardware unit is equipped with an accelerometer, gyroscope, pressure sensor, heart rate monitor, and short-range wireless communication transmitter as well as a power source. JSS has many features including but not limited to manual shot tracking ability for the wearer, manual court location tracking, ability to determine and convey when and where an ideal movement has occurred, a visual interface on JSS hardware to provide immediate feedback to the wearer on various conditions, adjustable sizing for various uses, moisture resistant, durable to withstand physical activity, ability to capture and report real-time data points to the wearer and coach, ability to compare data points for distinct users.

In use, a wearer user may calibrate the sleeve by putting the sleeve on the user's arm, selecting “calibrate” inside the JSS software, perform the desired athletic movements multiple times, which will be detected, recorded and stored by the JSS hardware, store the user's form of the athletic movement, and then the data on the athletic movement is transmitted to JSS and analyzed by JSS to produce data on the perfect performance of the athletic movement. Additionally, JSS will compare each subsequent athletic movement to the perfect performance data and report a value to the user which indicates how well the user is performing the athletic movement. When JSS detects the activity in the identified range, JSS stores the data points on that athletic movement for the particular user.

A JSS user needs an account to use the JSS. The user may identify themselves as either a coach or an athlete user. The athlete user will be prompted to enter data to generate an athlete user profile comprised of their first name, last name, program name, zip code, state, country, age, height, weight, gender, dominant hand (left or right), position played, whether the athlete user wants a private or public profile and a user photo (take photo, upload photo, or no photo). JSS tracks and monitors activity of the athlete user inside JSS and display statistics based on the data gathered from the athlete user's activity in JSS. After the creation of a profile, the athlete user will be prompted to calibrate their JSleeve hardware device by entering the MAC address (sensor address) for the upper arm and the forearm to register the hardware device with JSS software. JSS users may interact with one another based on whether or not they have been linked. A first JSS user may “follow” a second or later user to be updated on that user's activity inside JSS. The first JSS user may allow other JSS users (following users) to be updated on the data and activity of the first user, discretely remove a follower to limit the removed followers access to the activity of the first user, search filters to produce specific results, send and receive messages from other JSS users, record and monitor the win/loss record of each JSS user, competition against other JSS users based on data recorded and stored in JSS, the calculation of JSS user performance statistics stored for daily, weekly, monthly comparisons and to track improvement or decline in performance. Other features include shot recap data (including data on release time and pointers or other advice for improvement), fatigue level monitoring (Fresh, Working, Tired, Exhausted, Shots Made, For Accuracy) feedback based on analysis of JSS user data and comparison with ideal athletic movement data from JSS Library.

The athlete user's performance data points may optionally include data such as: shot count (stamina, accuracy, field goal), performance graphic designating quality of performance across three ranges based on percentage and location (make/miss, location, fatigue, basketball court with markings such as X and O). For the jump shot embodiment, the Sleeve is programmed with data points mechanics of the jump shot. Each shot programs into JSS app and matches to a collection of data points (for example, 150 data points), JSS recognizes those data points as the jump shot, JSS stores the user's 150 data points in JSS based on personal user profile. Subsequent user shots are measured against that user's own data points. JSS will calculate the user's percent accuracy to inform that user of their accuracy for that shot. Without JSS, the manual or technology free alternative is for a coach to observe the athlete and give tips based on that visual observation or for the athlete to self-identify issues based on poor performance and make a guess based on the athlete's own ability and technique.

Shot form replay within JSS displays an intentionally slowed down video replay of the user's arm while performing the activity of a jumpshot based on the data generated from the JSS sleeve and the library of forms stored within the JSS library for the user to easily digest and analyze for opportunities to improve efficiency for both right- and left-handed shooters. The Shot location tracking allows JSS to recognize when a shot has been taken by the user and the location on the court from which that shot was taken. Shots which enter the goal basket may be marked as O while missed shots may be marked as X, or another similar tracking system. JSS synchronizes shot timing with location data and references the court's dimensions as stored within the system and selected by the user during the session setup process to calibrate the JSS to the court dimensions. Basketball court dimensions may vary based on the level of play (high school, college, professional, international or other). Fatigue level tracking incorporates the heart rate monitoring sensor in the JSS to track and measure the user's heartrate while performing in a session against the user's resting heart rate to determine which customized range the user is in for every shot taken. The Virtual clipboard features a play diagramming software that allows users to draft plays and optimize results leveraging data from JSS for each athlete registered to their respective program for which the plays are being designed or drafted. The optimization of JSS provides data-based advice or guidance to the team or coach on the best player to perform each section of the desired play based on JSS data. Understanding the competitive landscape in JSS involves the leader boards and King of Court features, which allow users to compare themselves against other users within the JSS platform regarding the number of shots taken, shots made, level of accuracy and time spent within the JSS app. Coaches may also leverage this data to identify and follow users as part of a targeted recruiting process.

Other technical features may be readily apparent to those skilled in the art from the following figures and descriptions.

### BRIEF DESCRIPTION OF THE DRAWINGS

For a more complete understanding of this disclosure and its features, reference is now made to the following description, taken in conjunction with the accompanying drawings, in which:

FIG. 1 shows the “Sign up and login flow” of JSS.

FIG. 2 shows the “Sensors setup and session flow” of JSS.

FIG. 3 shows the “Followers and followings flow” of JSS.

FIG. 4 shows the “King of Court game flow” of JSS.

FIG. 5 shows the “Notifications flow” of JSS.

FIG. 6 shows the overview of JSS.

FIG. 7 is a sample sleeve component.

FIG. 8 is a sample sensor and power source setup.

### DETAILED DESCRIPTION OF DRAWINGS

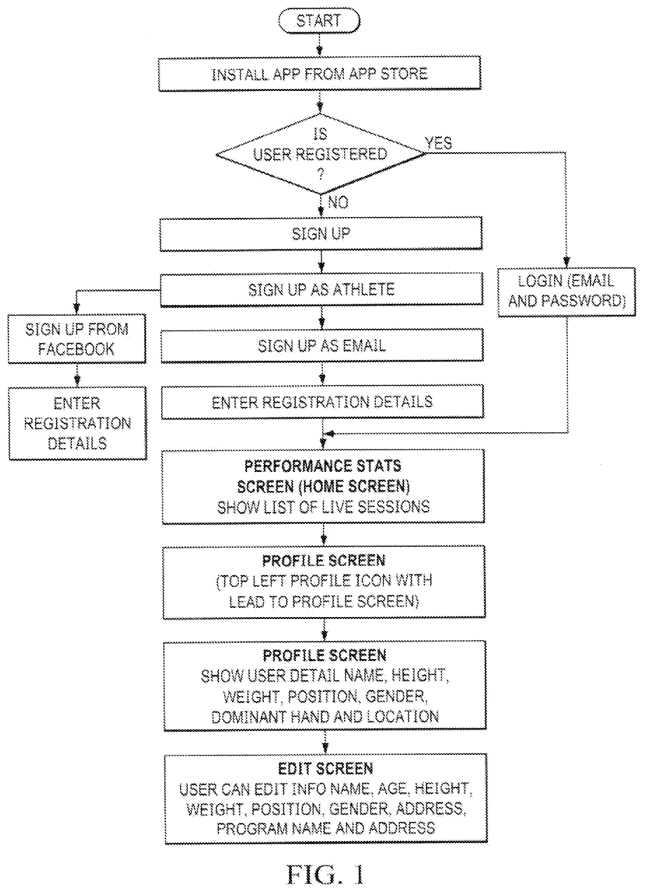

FIG. 1 shows the “Sign up and login flow” of JSS. The JSS user must install the JSS app on their device. If the user is registered, the user is prompted to sign in with email and password. An unregistered user will have to sign in and create a profile as either an athlete user or a coach user and provide the user's registration details. Once user registration details are entered in JSS, JSS directs the user to the performance stats home screen to show a list of live session and the user profile screen, which may include user height, weight, address, phone number, email address, sport position, gender, dominant hand and location, program or school name and address. The JSS user may enter the profile to update their own user profile information at any time.

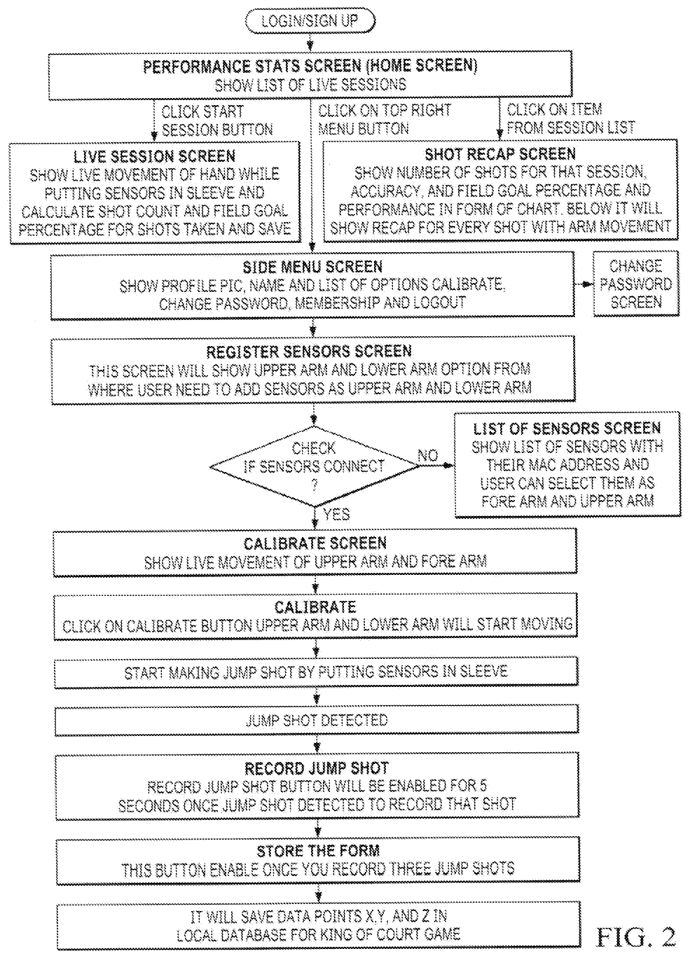

FIG. 2 shows the “Sensors setup and session flow” of JSS. Once a user logs in or signs up for JSS, JSS will present the user with the performance stats screen (home page), the user may click on the status session or button to start a live session screen, which shows the live movement of the user's hand or arm while the user puts the sensors in the compression sleeve and calculates the shot count and field goal percentage for shots taken. There may be at least two sensors on the sleeve, one on the upper arm and one on the lower arm. These data points are stored by JSS. The user may also click on an item from the session list (live session only), wherein JSS will present a shot recap screen, which shows the number of shots taken for the current session, accuracy and field goal percentage and performance in form of a chart. The recap for every shot and arm movement is included here. The JSS user may also select a side menu screen, which displays a profile picture, name, list of options such as calibration, change password, membership and logout. The register sensors screen will show the user where to add sensors to the upper arm and lower arm option. If the sensors connect, the JSS is calibrated and live movement of the upper arm and lower arm will be sent to JSS. Thereafter the user may select calibrate button on upper arm and/or lower arm to start moving. The user may perform the jump shot movement, which is detected by the sleeve. Once the jump shot or other athletic movement is detected, JSS will record the hump shot for several seconds, the data from the jump sot will be stored as the form for a jump shot within JSS. JSS will save the jump shot data points in a local database for playing the King of Court game.

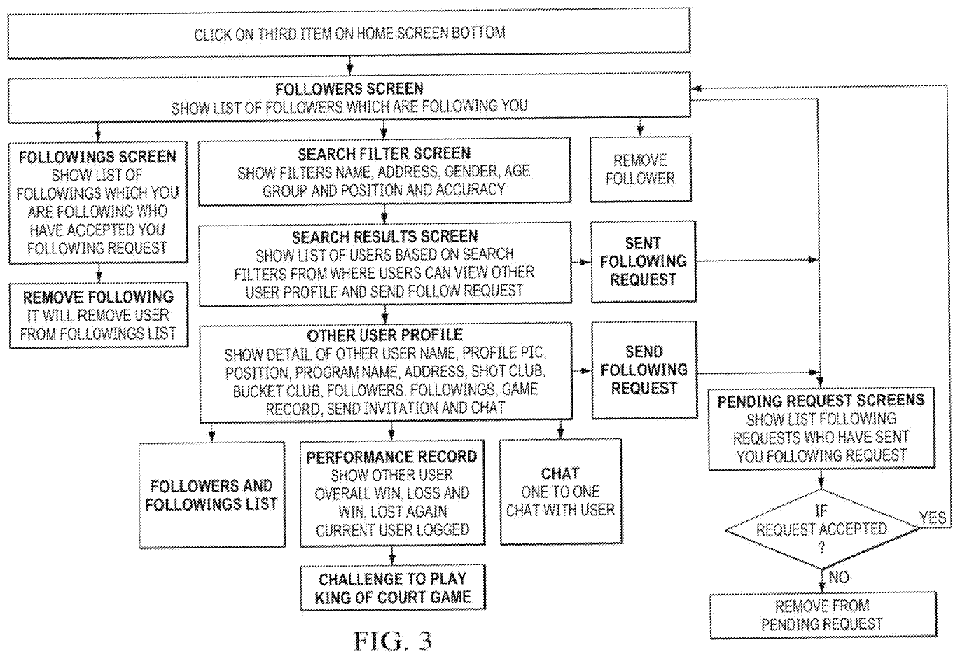

FIG. 3 shows the “Followers and followings flow” of JSS. A JSS user may determine which other JSS users are allowed to follow said user and access said user's data. A first user may select remove to prevent a later user from following the first user and accessing the first user's data. JSS displays a list of JSS users who have been accepted as followers and are allowed to access and view data of a first user. Users may filter their followers based on various characteristics such as name, address, gender, age, group, position, and accuracy rates. Users whose follow request has not been accepted as marked as pending and stored in the pending request screen. These users will be removed if the request is not accepted by the first user and added to the first user's followers if the request is accepted. JSS users may search all users with visible profiles and make follow requests to those users through the JSS platform. Once users are accepted and connected as following or followers, the users may view the profile of the other and access such user details as name, profile picture, position, program name, address, shot club, bucket club, followers, followings, game record, invitations and chat sessions. The user performance record shows the overall win rates and loss rates of the user. Users who are following each other may also request and accept a request to play a King of Court game. The winner and loser of the game will be recorded and stored in the user profile.

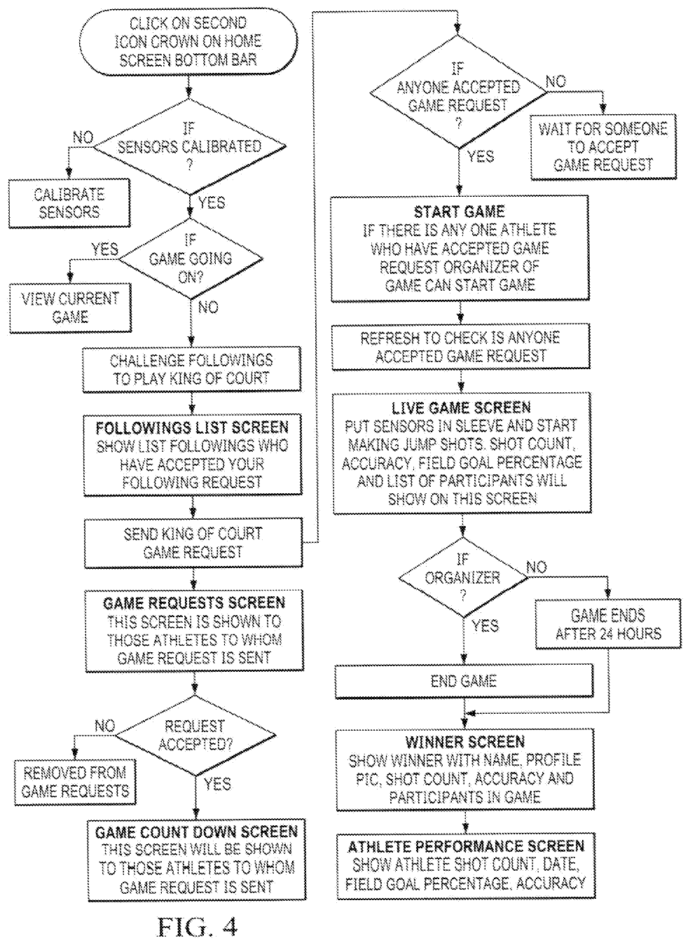

FIG. 4 shows the “King of Court game flow” of JSS. Before a King of Court game may begin, the selected users must calibrate their sensors in order for JSS to collect relevant data. Users may send requests to other user to join a game of King of Court in JSS. If the user accepts the game request, then the user is added to the session but if the user rejects or ignores the request, the request is removed from that user profile. Once the players for the game are set, the live game screen starts to record and store data from sleeves worn by players who are JSS users in the game session. The user who organizes the game has the option to end the game while the game will automatically end for the player users after 24 hours. Once the game ends, the winner screen will display the winner user's name profile, pic, shot count, accuracy rates, and the names/profiles of other game participants. The athlete performance screen will display each user's athlete shot count, date, field goal percentage, and accuracy rates.

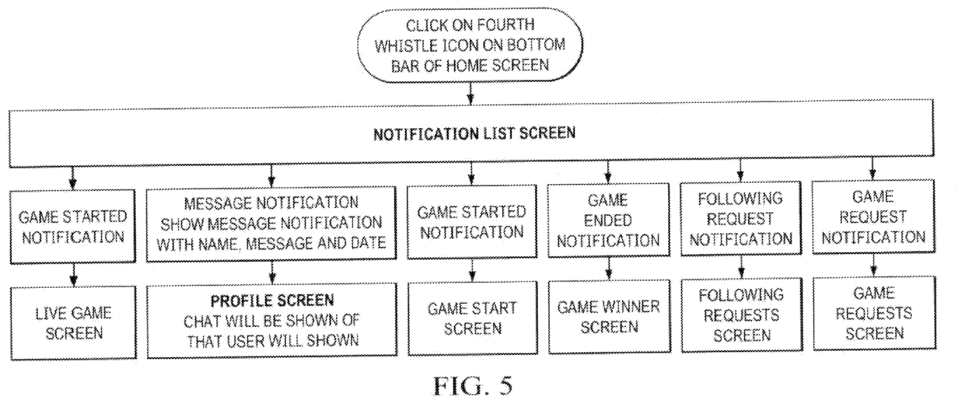

FIG. 5 shows the “Notifications flow” of JSS. Once a game request notification is sent in JSS, the request is displayed on the user game request screen. JSS users may receive notification of events such as game starts, message received in chat, game ends, request for follow received.

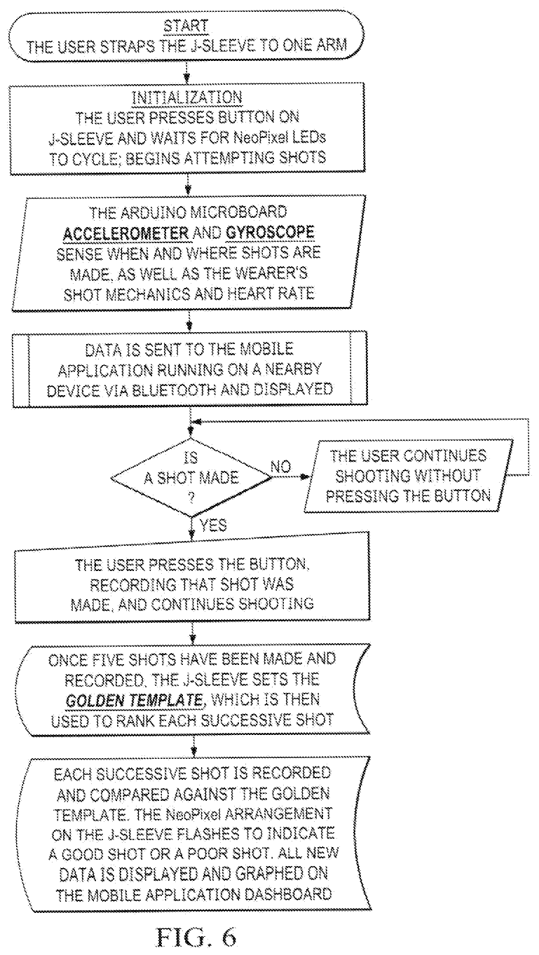

FIG. 6 shows the overview of JSS. The JSS user must secure the compression sleeve to the user's arm. The user then presses selects calibrate button to begin the calibration cycle. Upon calibration, the user will perform the athletic movement and data points will be collected included where the shot is taken, user's shot mechanics, and user's heart rate. The user data is sent to JSS via Bluetooth or other wireless communication network. If the shot or athletic movement was not successful, the user will continue shooting without pressing the button. If the shot or athletic movement was success, the user presses the button to activate JSS to record that a shot was made or completed while the user continued making shots. Once a predetermined number (three to five in this embodiment) of shots have been completed, the JSS sets the value for the “Golden Template”, which is later used by JSS to track and rank each successive user shot in comparison to the Golden Template. Each successive user shot is recorded and compared against the Golden Template, the JSS sleeve flashes to indicate a good shot or poor shot. All data collected from this exercise is displayed on the JSS mobile application dashboard.

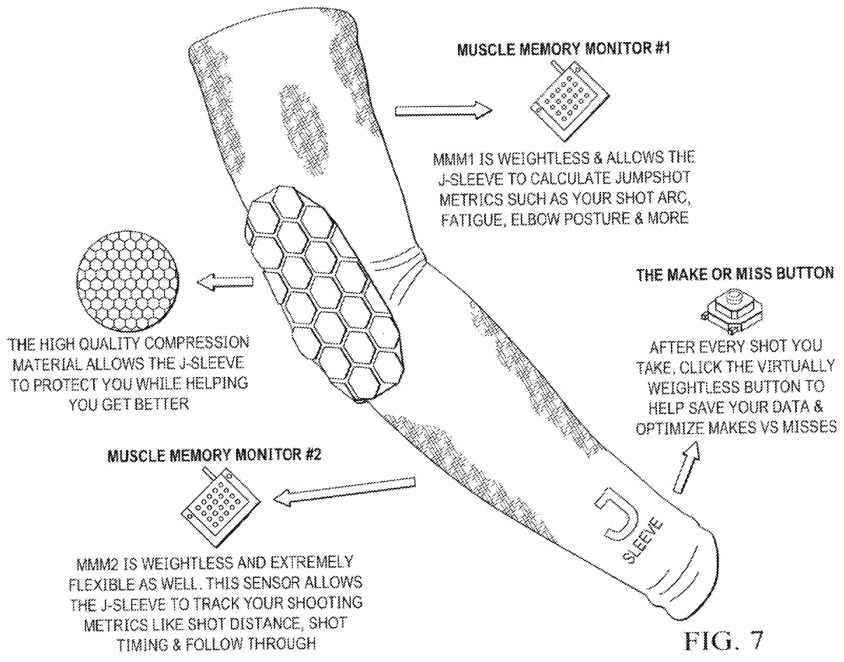

FIG. 7 is a sample embodiment of the JSS sleeve component. JSS includes a garment, such as a compression sleeve in this embodiment, which may be worn by the athlete user of JSS. Compression garments may be worn by athletes to improve performance, increase recovery times, improve circulation, and possibly prevent injury. Compression garments may be constructed of elastic synthetic fibers such spandex or nylon. The JSS may also include a “make or miss button” which may be pressed by the athlete user after every taken shot in order to save the athlete user performance data and track shots that the user makes and shots that the user misses. Further, the JSS includes at least two “muscle memory monitors”. The muscle memory monitor allows the JSS to calculate the metrics of the athlete user's physical activity, such as a jump shot and shot arc, and the heart rate monitor measures fatigue level of the athlete user by taking the actual heart rate relative to the resting heart and categorized the fatigue level into the categories such as Fresh, Working, Tired and Exhausted. Motion sensors also takes elbow posture as well as other metrics of the jump shot to provide diagnostic feedback to the end user, who may be a parent, coach, staff or other recipient user. Sensor synchronization along with court dimensions and JSS shot detection tracks shot distance to determine location for end user analysis and virtual clipboard optimization.

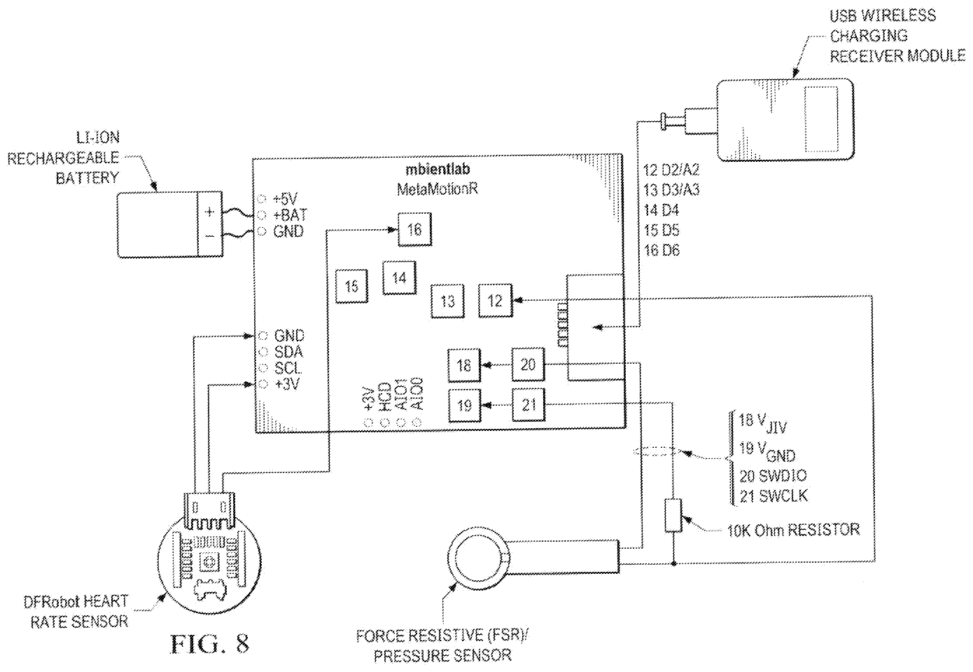

FIG. 8 is a sample embodiment of the JSS sensor and power source setup. JSS may be powered by a rechargeable battery that is connected to a charging pad. The wireless charging receiver is the primary power sources and is connected to the pressure sensor, motion sensor, and heart rate monitor.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure and the following claims.

What is claimed is:

1. A method for improving athletic performance of an athlete user comprising:
Attaching a garment system to an athlete user's body;
Calibrating said garment system to said athlete user's ideal movement;
Collecting athlete user performance data from a garment system worn on the body of an athlete user performing an activity;
Transmitting said athlete user performance data to an external processor over a wireless communication transmitter;
Analyzing said athlete user performance data of said athlete user performing said activity against said athlete user's ideal movement;
Developing recommendations for said athlete user based on the analysis of said athlete user performance data from said external processor;
Delivering said recommendations for said athlete user to a personal mobile device or visual interface on a mobile application;
Displaying said recommendations on a visual interface on said personal mobile device or said visual interface on a mobile application.

2. The method of claim 1, wherein said garment system is further comprised of a compression sleeve, accelerometer, gyroscope, pressure sensor, heartrate monitor, wireless communication transmitter, and a power source.

3. The method of claim 2, wherein said athlete user performance data is further comprised of a library of data points collected by said garment system and analyzed by said external processor.

4. The method of claim 3, wherein said external processor is further comprised of
a data collection unit consisting of a board, a power source, a response mechanism, sensors, and a personal mobile device, a visual display interface to provide instantaneous performance feedback to said athlete user and to provide said athlete user performance data to said recipient user.

5. The method of claim 4, wherein said external processor identifies an ideal movement for said athlete user through analysis of said library of data points collected for said athlete user.

6. The method of claim 5, wherein said recipient user may be selected from the group consisting of coach, staff, parent, and athlete user.

7. The method of claim 6, wherein said external processor develops recommendations for a recipient user on roster and plays to maximize performance of said athlete user and to increase scoring probability of said athlete user.

8. The method of claim 7, wherein said athlete user performance data may be selected from the group consisting of: name, age, height, weight, school, team, fatigue level, shot count, stamina, accuracy, field goal, location, athletic movement, ideal movement, shot form, shot technique, shot location tracking, moving heart rate, resting heart rate, and pulse.

9. The method of claim 1, wherein a first athlete user engages in competition with a second athlete user, wherein athlete user performance data for said first athlete user is tracked, analyzed and compared to athlete user performance data for said second athlete user by an external processor.

10. A method for selecting a team roster to maximize competition victories comprised of:
Collecting athlete user performance data for at least two athlete users from a garment system worn on the body of an athlete user performing an activity;
Analyzing said athlete user performance data of said athlete users performing said activity;
Reporting said athlete user performance data of said athlete users to an external processor;
Identifying an individual athlete user with the highest accuracy rating for each team position on a team roster;
Generating a recommendation for a team roster consisting of the most accurate athlete user for each player position on said team roster based on athlete user performance data.

11. A system for improving athletic performance comprised of
A garment system for the collection of data; and
A mobile computing device for the analysis and reporting of said data.

12. The system of claim 11, wherein said garment system is further comprised of a compression sleeve, accelerometer, gyroscope, pressure sensor, heartrate monitor, wireless communication transmitter, and a power source.

13. The system of claim 12, wherein said mobile computing device is further enabled with a second wireless communication transmitter to receive said data from said garment system.

14. The system of claim 13, wherein a user wearing said garment system records user performance data as said user performs movements, where said user performance data is recorded by said garment system, wherein said recorded user performance data is transmitted from said garment system to said mobile computing device, wherein said mobile computing device analyzes said recorded user performance data to product a user recommendation report for optimal performance based on said recorded user performance data.

15. The system of claim 14, wherein at least two athlete users each wear a garment system, wherein at least two athlete user recommendation reports for optimal performance are generated by at least two mobile computing devices based on recorded athlete user performance data transmitted from said garment systems, wherein a recipient user receives recorded user performance data for each user.
